(19) United States
(12) Patent Application Publication
(10) Pub. No.: US 2018/0002654 A1
(43) Pub. Date: Jan. 4, 2018

(54) DEVICE AND METHOD FOR SINGLE CELL SCREENING BASED ON INTER-CELLULAR COMMUNICATION

(71) Applicant: UNIST (ULSAN NATIONAL INSTITUTE OF SCIENCE AND TECHNOLOGY), Ulsan (KR)

(72) Inventors: Yoon Kyoung CHO, Ulsan (KR); Cedric BATHANY, Ulsan (KR); Jun Young KIM, Ulsan (KR); Devrim GOZUACIK, Istanbul (TR)

(21) Appl. No.: 15/536,748

(22) PCT Filed: Dec. 18, 2015
(86) PCT No.: PCT/KR2015/013959

§ 371 Date: Jun. 16, 2017

(30) Foreign Application Priority Data
Dec. 18, 2014 (KR) 10-2014-0183605
Mar. 11, 2015 (KR) 10-2015-0033711

(51) Int. Cl. C12M 1/34; G01N 33/48; C12M 1/12; C12M 3/06; G01N 33/543; G01N 33/50

(52) U.S. Cl. CPC C12M 41/46; G01N 33/54366; G01N 33/5005; C12M 1/34; C12M 25/02; C12M 23/16; G01N 33/48

(57) ABSTRACT

A device for single-cell analysis according to an embodiment of the present invention comprises: a substrate; a gap between the substrate and porous membrane which is a space for culture medium; and a porous membrane formed on having a pore capable of isolating a second cell into single cell units.

A method for single-cell analysis according to an embodiment of the present invention comprises: Culturing a first cell in a culture medium on a bottom side of porous membrane; Applying a sample including a second cell on a porous membrane in a culture medium; Isolating the second cell into single cell units in a pore existing in the porous membrane with a external force such as agitation and gravitational force; Generating an interaction situation between the first cells and the single cell-level second cell; Analyzing a cellular phenomena of the first cell or the second cell.

### FIELD OF THE INVENTION

The present invention relates to a device and a method for single cell level screening based on interaction among single cell and neighboring multiple cells.

### DESCRIPTION OF THE RELATED ART

In cellular microenvironment, cells give and receive messages with its environment and with itself via cytokine signals and/or direct contact affecting cellular phenotypes. For this significance, in vitro platform for cell-to-cell interaction was actively developed in a form of 2-D or 3-D platform to mimic and to investigate the interactions between cell populations. But it can obtain only average results from many numbers of cells. This in turn motivates the development of complementary in vitro platform of single cell isolation and its analysis.

Single cell isolation techniques have been developed by using microwell arrays, traps using hydrodynamic fluid control, dielectrophoresis and surface micropatterning etc. Using these single cell isolation techniques, they used these single cell isolation techniques in various application such as analysis of heterogeneous cellular phenotype, paracrine factor secretion and DNA repair capacities with different genetic backgrounds.

Specifically, single cell pairing techniques have been highlighted because it can achieve not only the spatiotemporal control of cellular interaction but also make a special situation for single cell level interaction. The application includes cell migration, proliferation patterns of stem cell, and heterogeneous dynamics of CD8 T cells through interaction with lymphocyte. It can provide a single cell level resolution in resolving stochastic cellular behavior in large populations, which helps to understand the cell dynamics and to achieve better statistical data of intercellular signaling mechanisms unlike conventional bulk system. The previously reported single cell pairing method has a limit which focuses on only single cell and single cell interaction. There is a gap between the in-vitro single cell and single cell interaction chips and in-vivo cellular microenvironment. For example, tumor cells are situated in a microenvironment surrounded by multiple stromal cells and interact each other.

### CONTENTS OF THE INVENTION

### Problem to be Solved

The purpose of the present invention is to provide a device and a method for screening cells in a single cell level based upon intercellular communication between single cell and neighboring multiple cells.

### Means for Solving Problem

A device for single-cell analysis according to an embodiment of the present invention comprises: a substrate; a gap between membrane and substrate and capable of culturing a first cell; and a porous membrane having a pore capable of isolating a second cell into single cell units.

A gap between the porous membrane and the substrate may be 1 to 100 μm.

The porous membrane may be selected from polymeric or inorganic materials.

The porous membrane may be made by forming a pore in a polymeric membrane through a soft lithography method.

The porous membrane may be a photosensitive polymeric material.

The porous membrane may be made by forming a pore in a photosensitive polymeric membrane through a lithography method.

A diameter of the pore may be 1 to 100 μm.

The porous membrane may have pores of 10² to 10⁶ holes/cm².

The porous membrane may have pores and a gap between the pores may be 1 μm to 10 mm.

A method for single-cell analysis according to an embodiment of the present invention comprises: Culturing a first cell in a culture medium on a bottom side of porous membrane; Applying a sample including a second cell on a porous membrane in a culture medium; Isolating the second cell into single cell units in a pore existing in the porous membrane with a external force such as agitation and gravitational force; Generating an interaction situation between the first cells and the single cell-level second cell; Analyzing a cellular phenomena of the first cell or the second cell.

The first cell may be a fibroblast cell and the second cell may be a tumor cell.

A gap between porous membrane and substrate may be 1 to 100 μm.

A concentration of the first cell may be 1×10⁵ to 1×10⁷ cells/mL.

A concentration of the second cell in the sample may be (a number of pores in a porous membrane×1) to (a number of pores in a porous membrane×10,000) cells/mL or 1×10² to 1×10¹⁰ cells/mL.

When applying the external force such as gravitation force, stirring may be performed at the same time. Moreover, the stirring may be performed for 1 minute to 1 hour at 0 to 500 rpm.

A diameter of the pore may be 1 to 100 μm.

The porous membrane may have pores of 10² to 10⁶ holes/cm².

The porous membrane may have pores and a gap between the pores may be 1 μm to 10 mm.

The interaction may be generated by contact and paracrine communication between the first cells and the second cell for 1 hour to 7 days.

The analyzing of cellular activities of the first cell or the second cell may further comprise monitoring the cellular activities of the first cells or the second cell.

The analyzing of cellular activities of the first cells or the second cell may further comprise obtaining and analyzing the first cells.

The analyzing of cellular activities of the first cells or the second cell may further comprise capturing and analyzing the second cell.

### Effects of the Invention

A cellular phenomenon monitoring and analysis at single cell units according to the single cell-to-bulk cells interaction may be easily performed. A gene analysis can be available as well as a visual analysis at single cell units.

### BRIEF DESCRIPTION OF THE DRAWINGS

FIG. 1 is a schematic view illustrating a device for single-cell analysis according to an embodiment of the present invention.

FIG. 2 is a close-up photograph of a porous membrane with pores.

FIG. 3 is a schematic view illustrating a device for single-cell analysis when culturing a first cell on bottom side of a porous membrane.

FIG. 4 is a schematic view illustrating a device for single-cell analysis when isolating a second cell into a pore of a porous membrane.

FIG. 5 is a photograph of a device for single-cell analysis according to an embodiment of the present invention.

FIG. 6 is a flowchart of a method for single-cell analysis according to an embodiment of the present invention.

FIG. 7 is a close-up photograph of a tumor cell isolated in a pore.

FIG. 8 is a close-up photograph of screening a fibroblast existing on a bottom side of porous membrane and generating an autophagy phenomenon by an interaction with an isolated single tumor cell.

FIG. 9 is a graph to explain a monitor performance of the present invention, which holes with single cell have a significant difference between empty hole in case of an percentage of autophagy phenomenon in fibroblasts.

FIG. 10 is a photograph of isolation of a single tumor cell from a pore and genomic result using this single tumor cell.

### DETAILED DESCRIPTION

The terminology used in the specification is for the purpose of referring to particular embodiments by way of example only. Thus, the terminology is not intended to be limiting of the present invention. The singular forms used in the specification include the plural forms, unless the context clearly dictates otherwise. The term “comprising” used in the specification specify the specific characteristics, regions, integers, steps, operations, elements, and/or components, but do not preclude the presence or the addition of other specific characteristics, regions, integers, steps, operations, elements, and/or components.

Unless otherwise defined, all terms including technical and scientific terms used herein have the same meaning as commonly understood by those skilled in the art of the present invention. The terms defined in commonly used dictionaries are additionally interpreted as having a meaning that is consistent with the relevant technical literature and current disclosure, but are not interpreted in an idealized or overly formal sense unless expressly defined herein.

Hereinafter, embodiments of the present invention will be described in detail. However, it is for illustrative purpose only and not meant to limit or otherwise narrow the scope of the present invention. Therefore, the present invention will only be defined by the appended claims.

FIG. 1 schematically shows a device for single-cell analysis according to an embodiment of the present invention. The device for single-cell analysis in FIG. 1 is intended to be merely illustrative of the present invention, and the present invention is not limited thereto. Thus, a device for single-cell analysis may be modified in various ways.

As shown in FIG. 1, a device for single-cell analysis according to an embodiment of the present invention comprises: a substrate; a gap between the substrate and porous membrane which is a space for culture medium; and a porous membrane formed on having a pore capable of isolating a second cell into single cell units. The second cell (200) isolated in the pore (31) as a single cell performs an interaction with the first cell (100) cultured in the culture medium, and then the second cell (200) together with the porous membrane (30) is separated from the first cell (100). Thus, the second cell (200) may be analyzed as single cell units. As a result, an analysis of single cell units according to a single cell-to-bulk cells interaction may be easily performed. Moreover, a gene analysis can be available as well as a visual analysis at single cell units.

A gap between the porous membrane (30) and the substrate may be 1 to 100 μm. If the gap between the porous membrane (30) and the substrate is too narrow, a culture of the first cell (100) on the bottom side of porous membrane is difficult. If the gap between the porous membrane (30) and the substrate is too big, the second cell (200) is not isolated in the pore (31), but passed through the gap between the porous membrane (30) and the substrate. In particular, the gap between the porous membrane (30) and the substrate may be 1 to 100 μm.

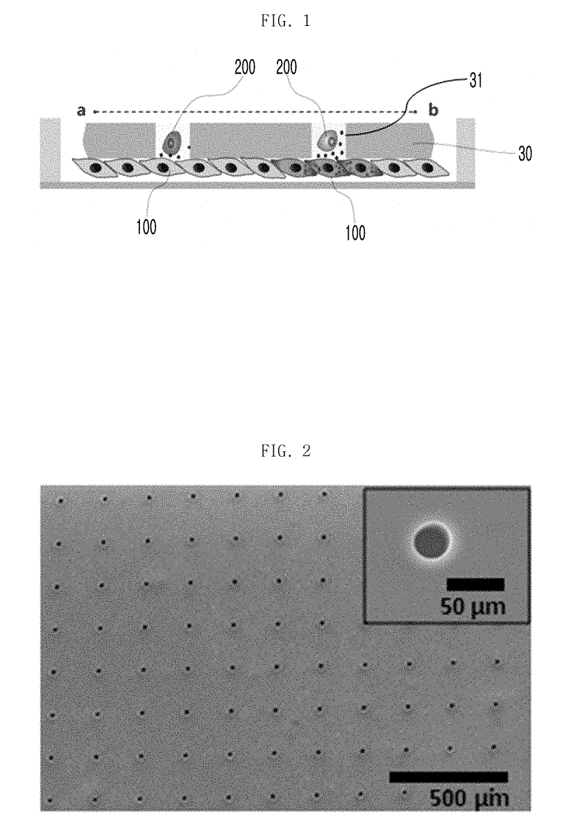

The porous membrane (30) may be a polymeric material. More specifically, the polymeric material are polymethyl(meth)acrylate (PMMA), polydimethylsiloxane (PDMS), polycarbonate (PC), polyethylene terephthalate (PET), polypropylene (PP), and the like. When using a polymeric material as the porous membrane (30), a soft lithography method may be used to form the pore (31) in the porous membrane (30). When using a photosensitive polymeric material as the porous membrane (30), a lithography method may be used to form the pore (31) in the porous membrane (30). Describes an example of the process of forming the pore (31) in the porous membrane (30) through the soft lithography method is as follows. Deposit the photoresist on silicon wafer. Make a pattern using photolithography, Add pre-cured PDMS and cured, Peel away PDMS master, Do RIE treatment with CHF3, Inject pre-cured PDMS into the gap between PDMS master and glass, Release PDMS membrane where the pore (31) is formed. A close-up photograph of the porous membrane (30) with a pore (31) is described in FIG. 2.

Slide glass or polydimethylsiloxane (PDMS) may be used as the substrate. A device for single-cell analysis may be obtained by using a soft etching method (soft lithography) on the substrate to secure a space, by processing a surface of the substrate. The method stated above is only one example to prepare the device for single-cell analysis and may vary depending on needs.

A diameter of the pore (31) formed in the porous membrane (30) may be 1 to 100 μm. If the diameter of the pore (31) is too small, the second cell (200) is difficult to be isolated in the pore (31). If the diameter of the pore (31) is too big, the second cell (200) may be not isolated as single cell units.

The porous membrane (30) may have pores of 10² to 10⁶ holes/cm². If the pore (31) is too small, the amount of the second cell (200) for an analysis may become too small. If the pore (31) is too large, there is a problem that a device for analysis of the second cell (200) may become large.

A gap between the pores (31) formed in the porous membrane (30) may be 1 μm to 10 mm. If the gap between the pores (31) is too narrow, an interaction between the neighboring first cells (100) cultured in the culture medium occurs. Thus, an analysis of a cellular phenomenon caused by an interaction between the first cell (100) and the second cell (200) may be difficult. If the gap between the pores (31) is too wide, there is a problem that a device for analysis of the second cell (200) may become large.

A device for single-cell analysis according to an embodiment of the present invention may further comprise a reservoir, porous membrane, gap between porous membrane and substrate.

FIG. 3 schematically describes a culture of the first cell (100) on the bottom side of the membrane (20).

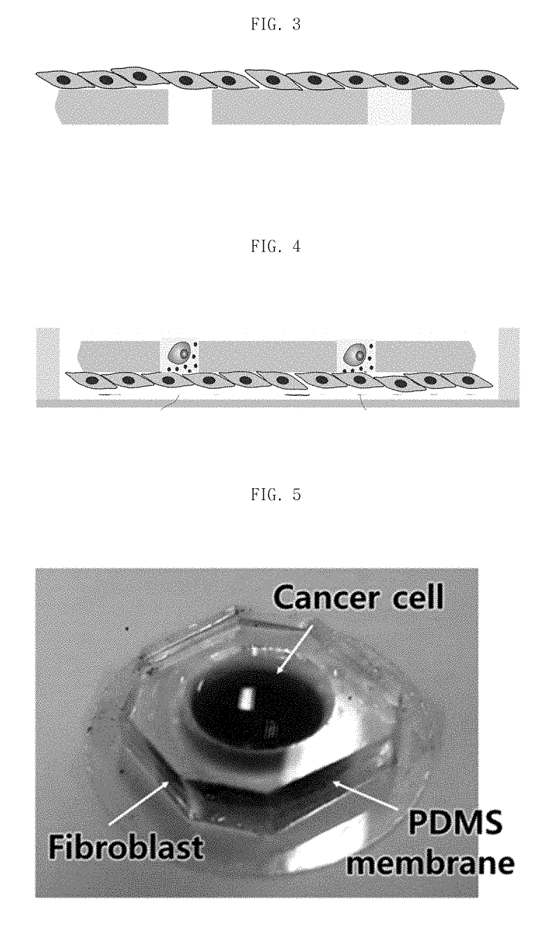

FIG. 4 schematically describes an isolation of the second cell (200) in the pore (31) of the porous membrane (30). By applying the external forces such as agitation and gravitational force, the second cell (200) is isolated in the pore (31) at single cell units.

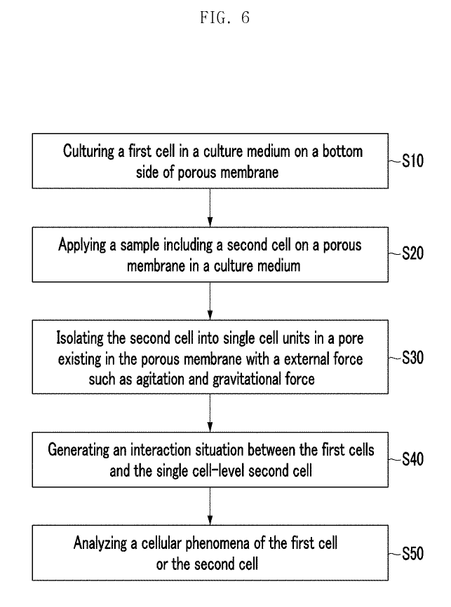

FIG. 5 describes a photograph of a device for single-cell analysis according to an embodiment of the present invention.

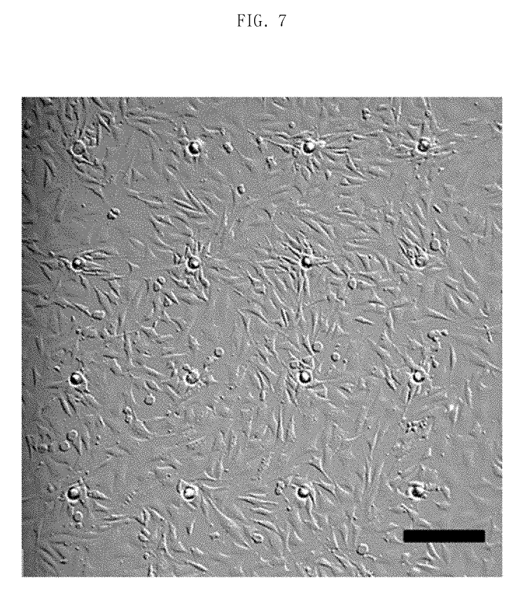

FIG. 6 schematically describes a flowchart of a method for single-cell analysis according to an embodiment of the present invention. The flowchart of a method for single-cell analysis in FIG. 6 is intended to be merely illustrative of the present invention, and the present invention is not limited thereto. Thus, a method for single-cell analysis may be modified in various ways.

As shown in FIG. 6, a method for single-cell analysis comprises: culturing a first cell (100) on the bottom side of porous membrane (20) formed on the gap between a porous membrane and a substrate (S10): applying a sample including a second cell (200) on a porous membrane (30) in the culture medium (20) (S20); isolating the second cell (200) into single cell units in a pore (31) existing in the porous membrane (30) with a external force such as agitation and gravitational force (20) (S30); generating an interaction situation between the first cells (100) and the single cell-level second cell (200) (S40);and analyzing a cellular activity of the first cells (100) or the second cell (200).

First, in step S10, the first cell (100) is cultured on the bottom side of a porous membrane. A thickness of a gap between the substrate and the porous membrane may be 1 to 100 μm. If the gap between the porous membrane (30) and the substrate is too narrow, a culture of the first cell (100) in the culture medium is difficult. If the gap between the porous membrane (30) and the substrate is too wide, the second cell (200) is not isolated in the pore (31), but passed through the culture medium.

When culturing the first cell (100) on the bottom side of the porous membrane, the medium containing first cell (100) is put on the bottom side of the porous membrane for few hours (20) and the porous membrane is attached with substrate, which gap between the porous membrane and substrate can supply the nutrient. A concentration of the first cell (100) may be 1×10⁵ to 1×10⁷ cells/mL.

FIG. 3 schematically describes a culture of the first cell (100) on the bottom side of the porous membrane (20). The medium containing first cell (100) is put on the bottom side of the porous membrane for few hours (20) and the porous membrane is attached with substrate

If a cell can induce an interaction with the second cell (200), the cell may be used as the first cell (100) without restriction. In particular, the first cell may be a fibroblast cell.

In step S20, a sample including the second cell (200) is applied on the porous membrane (30) with external forces such as agitation and gravitational forces. The sample includes the second cell (200) as well as a medium, wherein a concentration of the second cell (200) in the sample may be (a number of pores in a porous membrane×1) to (a number of pores in a porous membrane×10,000) cells/mL or 1×10² to 1×10¹⁰ cells/mL. If the concentration of the second cell (200) is too low, the efficiency of the analysis may be reduced because empty pores (31) in which the second cell (200) is not isolated become a lot. If the concentration of the second cell (200) is too high, the second cell is difficult to be isolated in the pore (31) as single cell units.

If a cell can induce an interaction with the first cell (200) to analyze changes of cellular activities after the interaction, the cell may be used as the second cell (200) without restriction. In particular, the second cell may be a tumor cell.

In step S30, the second cell (200) is isolated into single cell units in a pore (31) existing in the porous membrane (30) by applying external forces such as agitation and gravitational forces.

FIG. 4 schematically describes an isolation of the second cell (200) in the pore (31) of the porous membrane (30). By applying external forces such as agitation and gravitational forces in the direction of the arrow, the second cell (200) is isolated in the pore (31) at single cell units.

When applying external forces such as agitation and gravitational forces, a sample including the second cell (200) in the reservoir is directed into the pore (31) formed on the porous membrane (30). Untrapped second cells are washed and then, only the trapped second cell (200) is isolated in the pore (31) in a single-cell state. At this time, the applied agitation velocity may be 0 to 200 rpm. For example, the stirring may be performed by a method of putting the device for single-cell analysis on a shaker. The stirring may be performed for 1 minute to 1 hour at 10 to 500 rpm. If the agitation velocity is too slow, the second cells are hard to spread. If the agitation velocity is too fast, the second cells (200) tend to gather on edge part.

When applying the agitation force, different number of second cells input may be performed at the same time. The number of second cells input may be varied from 1*number of total pores to 1,000*number of total pores. If a input number of second cells is too low, there may be a problem in efficiency of single cell entrapment is low. If a input number of second cells is too many, a percentage of multiple cell entrapment is increased Thus, there is a problem that the cell is isolated only to a specific part in single cell level.

A diameter of the pore (31) isolating the second cell (200) may be 1 to 100 μm. If the diameter of the pore (31) is too small, the second cell (200) is difficult to be isolated in the pore (31). If the diameter of the pore (31) is too wide, the second cell (200) may be not isolated as single cell units.

The porous membrane (30) may have pores of 10² to 10⁶ holes/cm². If the pore (31) is too small, there is a problem that the amount of the second cell (200) for an analysis may become too small. If the pore (31) is too large, the efficiency of the analysis may be reduced because empty pores (31) in which the second cell (200) is not isolated become a lot.

A gap between the pores (31) isolating the second cell (200) may be 1 μm to 10 mm. If the gap between the pores (31) is too narrow, an interaction between the neighboring first cells (100) cultured on the bottom side of porous membrane occurs. Thus, an analysis of a cellular phenomenon caused by an interaction between the first cell (100) and the second cell (200) may be difficult. If the gap between the pores (31) is too wide, there is a problem that a device for analysis of the second cell (200) may become large.

In step S40, an interaction is generated by contact or paracrine factor between the first cell (100) and the second cell (200). At this time, the interaction is generated for 1 hour to 7 days. For example, the interaction may be caused by directly contacting between a tumor cell and a fibroblast cell or by an indirect paracrine factor.

A method of analyzing may be screening of the cell activity changed by the interaction between the first cells (100) or the second cells (200), or capturing and analyzing the second cell (200) completing the interaction. The second cell (200) completing the interaction exists inside of the pore (31) of the porous membrane (30) in an isolated state, so single cell units of the second cell (200) may be analyzed by separating the second cell (200) form the first cell (100).

In particular, the interaction between the first cell (100) and the second cell (200) may be analyzed by a green marker previously inserted in the first cell (100). Moreover, a gene analysis may be performed by obtaining the second cell (200) as single cell units through a single cell picker (Kuiqpick) and analyzing the obtained second cell (200) through a single cell genetic analysis device (Biomark HD).

Therefore, the visual analysis as well as the gene analysis of single cell units can be available.

Below a preferred embodiment of the present invention and comparative examples will be described. However, embodiment stated below is just an embodiment of the present invention, so the present invention is not limited thereto.

### Example 1

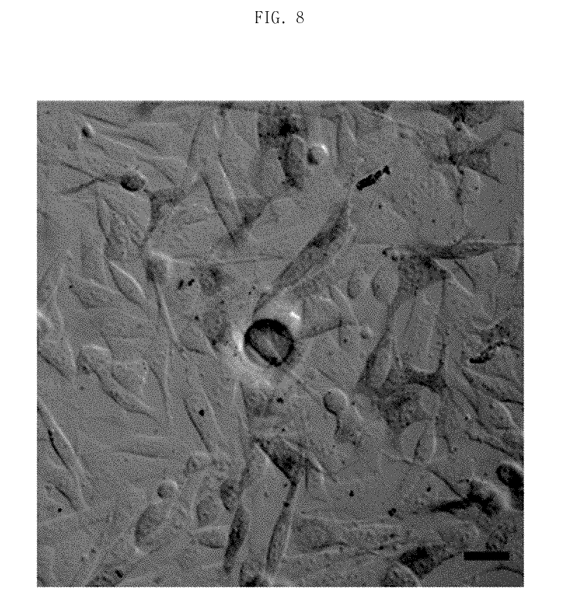

A porous membrane having 5,000 pores whose pore size is 30 μm was prepared by using a Polydimethylsiloxane (PDMS). A Polydimethylsiloxane (PDMS) coated substrate was prepared as a substrate having 5 μm thickness. The substrate was used as a space of culture medium. FIG. 7 is a close-up photograph of a tumor cell isolated in a pore. A tumor cell was isolated in the pore as single cell units by applying a sample including a tumor cell on the porous membrane, by applying 10,000 input number of second cells, and by stirring for 5 minutes at 100 rpm.

Table 1 shows yield efficiency obtained by organizing a number ratio of the tumor cell isolated in the pore against a number of the tumor cell applied on the porous membrane.

FIG. 8 is a close-up photograph of screening a fibroblast existing on a bottom side of porous membrane and generating an autophagy phenomenon by an interaction with an isolated single tumor cell.

We observed whether a cell change of a fibroblast cell occurs by performing interaction between a tumor cell and a fibroblast cell for 6 hours. Thus, we can found that there was an interaction with the second cell isolated in the pore and the first cell.

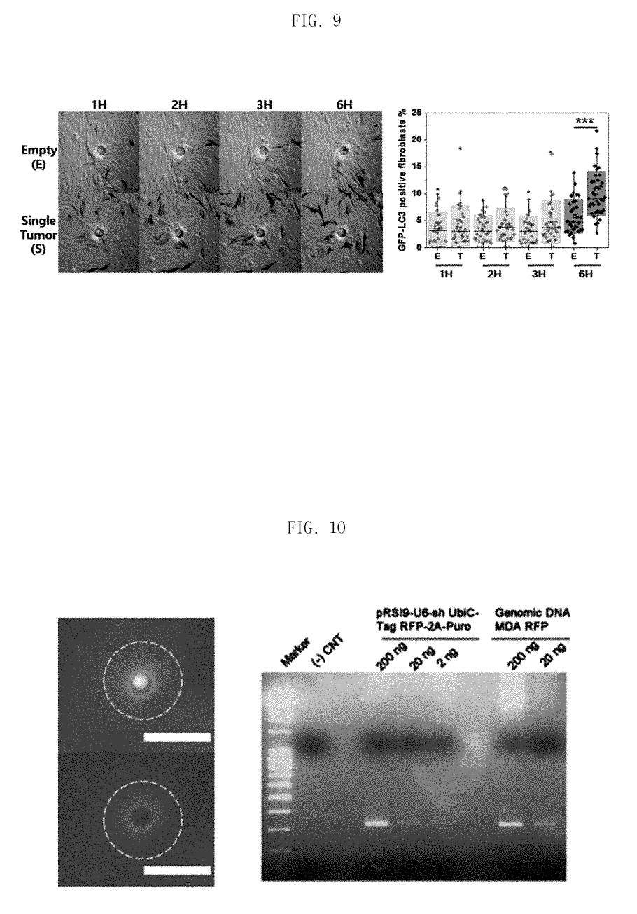

FIG. 9 is a graph to explain a monitor performance of the present invention, which holes with single cell have a significant difference between empty hole in case of an percentage of autophagy phenomenon in fibroblasts.

Table 2 shows comparison between empty holes and holes with single tumor in case of autophagy activation percentage in fibroblasts.

FIG. 10 is a photograph of isolation of a single tumor cell from a pore and genomic result using this single tumor cell. We observed that the proteins extracted from isolated single cell can be used to do gene analysis.

### Example 2

The stirring speed was adjusted to 0 rpm. The rest of the experiments were performed in the same manner as in Example 1.

### Example 3

The stirring speed was adjusted to 200 rpm. The rest of the experiments were performed in the same manner as in Example 1.

### Example 4

The number of second cells input was adjusted to 5,000. The rest of the experiments were performed in the same manner as in Example 1.

### Example 5

The number of second cells input was adjusted to 20,000. The rest of the experiments were performed in the same manner as in Example 1.

TABLE A

| | Number of | Stirring | Stirring | Yield |
|---|---|---|---|---|
| | second cells | speed | time | efficiency |
| | input | (rpm) | (min) | (%) |
| Example 1 | 10,000 | 100 | 5 | ~50 |
| Example 2 | 10,000 | 0 | 5 | ~40 |
| Example 3 | 10,000 | 200 | 10 | ~35 |
| Example 4 | 5,000 | 100 | 5 | ~40 |
| Example 5 | 20,000 | 100 | 5 | ~35 |

As shown in Table 1, a cell may be isolated in the pore as single cell units by adjusting various conditions such as the amount of number of second cells input, stirring speed, stirring time.

The present invention is not limited to the embodiments, and may be prepared in different forms. Those skilled in the art of the present invention can understand that it can be embodied in other specific forms without departing from its spirit or essential characteristics. Therefore, the described embodiments are to be considered just as illustrative and not restrictive in all respects.

### DESCRIPTION OF REFERENCE NUMERALS

| | | | |
|---|---|---|---|
| 20: | culture medium | 30: | porous membrane |
| 31: | pore | 100: | first cell |
| 200: | second cell | | |

What is claimed is:

1. A device for single-cell analysis comprising:
a substrate;
a gap between the substrate and a porous membrane which is space for culture medium; and
a porous membrane having a pore capable of isolating a second cell into single cell units.

2. The device of claim 1,
wherein a gap between the porous membrane and the substrate is 1 to 100 μm.

3. The device of claim 1,
wherein the porous membrane is made of selected from polymeric or inorganic materials.

4. The device of claim 3,
wherein the porous membrane is made of a photosensitive polymeric material.

5. The device of claim 3,
wherein the porous membrane is made by forming a pore in a polymeric membrane through a soft lithography method.

6. The device of claim 4,
wherein the porous membrane is made by forming a pore in a photosensitive polymeric membrane through a lithography method.

7. The device of claim 1,
wherein a diameter of the pore is 1 to 100 μm.

8. The device of claim 1,
wherein the porous membrane has pores of 10² to 10⁶ holes/cm².

9. The device of claim 1,
wherein the porous membrane has pores and a gap between the pores is 1 μm to 10 mm.

10. A method for single-cell analysis comprising:
Culturing a first cell in a culture medium on a bottom side of porous membrane; Applying a sample including a second cell on a porous membrane in a culture medium; Isolating the second cell into single cell units in a pore existing in the porous membrane with an external force such as agitation and gravitational force; Generating an interaction situation between the first cells and the single cell-level second cell; Analyzing cellular phenomena of the first cell or the second cell.

11. The method of claim 10,
wherein the first cell is a fibroblast cell and the second cell is a tumor cell.

12. The method of claim 10,
wherein a thickness of the culture medium is 1 to 100 μm.

13. The method of claim 10,
wherein a concentration of the first cell is 1×10⁵ to 1×10⁷ cells/mL.

14. The method of claim 10,
wherein when applying the second cells, stirring is performed at the same time.

15. The method of claim 10,
wherein a concentration of the second cell in the sample i (a number of pores in a porous membrane×1) to (a number of pores in a porous membrane×10,000) cells/mL or 1×10² to 1×10¹⁰ cells/mL.

16. The method of claim 14,
wherein the stirring is performed for 1 minute to 1 hour at 10 to 500 rpm.

17. The method of claim 10,
wherein a diameter of the pore is 1 to 100 μm.

18. The method of claim 10,
wherein the porous membrane has pores of 10² to 10⁶ holes/cm².

19. The method of claim 10,
wherein the porous membrane has pores and a gap between the pores is 1 μm to 10 mm.

20. The method of claim 10,
wherein the interaction is generated by contact or paracrine factors between the first cell and the second cell for 1 hour to 7 days.

21. The method of claim 10,
wherein the analyzing a cell activity of the first cell or the second cell further comprises screening the cell activity of the first cell or the second cell.

22. The method of claim 10,
wherein the analyzing a cell activity of the first cell or the second cell further comprises capturing and analyzing the second cell.
